(19) United States
(12) Patent Application Publication
(10) Pub. No.: US 2006/0008886 A1
(43) Pub. Date: Jan. 12, 2006

(54) Portable shooting house

(76) Inventor: Mark C. Lippe, Moss Point, MS (US)

Correspondence Address: WOOD, NGO & EISENBERG, PLLC, 2121 Eisenhower Ave, Suite 200, Alexandria, VA, 22314, US

(21) Appl. No.: 11/176,273

(22) Filed: Jul. 8, 2005

Related U.S. Application Data
(60) Provisional application No. 60/585,864, filed on Jul. 8, 2004.

(51) Int. Cl. C12P 13/04

(52) U.S. Cl. 435/106

(57) ABSTRACT

A hunting house, comprising: an elongated tongue 150, a removable axle 350, a framework 700, and a camouflaged exterior 650 fitted over said framework 700.

### CROSS-REFERENCE TO RELATED APPLICATIONS

This application claims the benefit of priority from U.S. Provisional Patent Application Ser. No. 60/585,864, filed 07-08-2004, the entire contents of which are incorporated by reference herein.

### STATEMENT REGARDING FEDERALLY SPONSORED RESEARCH OR DEVELOPMENT

Not Applicable.

### FIELD OF THE INVENTION

This invention relates to the field of hunting shelters and more particularly to a portable shooting house that requires minimum on-site assembly.

### BACKGROUND OF THE INVENTION

As noted in U.S. Pat. No. 6,286,269 issued Sep. 11, 2001 to G. M. Marcum, it has long been recognized that hunters need some protection from inclement weather and should remain unnoticed as they await their prey. However, the Marcum '269 hunting shelter come shooting house requires a lot of effort and time to assemble. For example, before using the Marcum '269 house a hunter is required to: position four side panels, then affix three of the side panels to a floor and adjoining side panels, then position a top panel on top of the shooting house, then affix the fourth side panel to the floor and adjoining side panels; and then next affix the top panel to all side panels; wherein positioning the top panel includes: bringing the top panel through a door, then aligning the top panel along the body diagonal of the shooting house, then raising the top panel from the body diagonal to the top of the shooting house, and finally matching an attached lip on the top panel with an attached lip on each side panel. In addition, a hunter has to transport the Marcum '269 shooting house in pieces and then unpack the pieces before assembling the pieces to provide the shooting house.

In some hunting areas the hunting management does not allow hunting or shooting houses to be left up overnight. Thus, there is a need for a shooting house that is easily transported to a hunting site and which does not require extensive effort to assemble on site.

### SUMMARY

A hunting house, comprising: an elongated tongue 150, a removable axle 350, a framework 700, and a camouflaged exterior 650, fitted over said framework 700.

### DESCRIPTION OF THE INVENTION

This invention relates to the field of hunting shelters and more particularly to a portable shooting house that requires minimum on-site assembly. The shooting house of the present invention is essentially towed in one piece to a hunting site by a motor vehicle with a hitch point; the shooting house is unhitched, a removable axle removed, and the shooting house raised to an upright position for immediate use. Only if there is a requirement to raise the shooting house above the ground is there a requirement to attach legs and optionally an anchoring system to anchor the raised shooting house to the ground. Attachments such as legs, ladders and anchoring system are towed with the shooting house. The terms “stand”, “shooting house”, “device 100”, and “hunting house” are hereinafter regarded as equivalent terms.

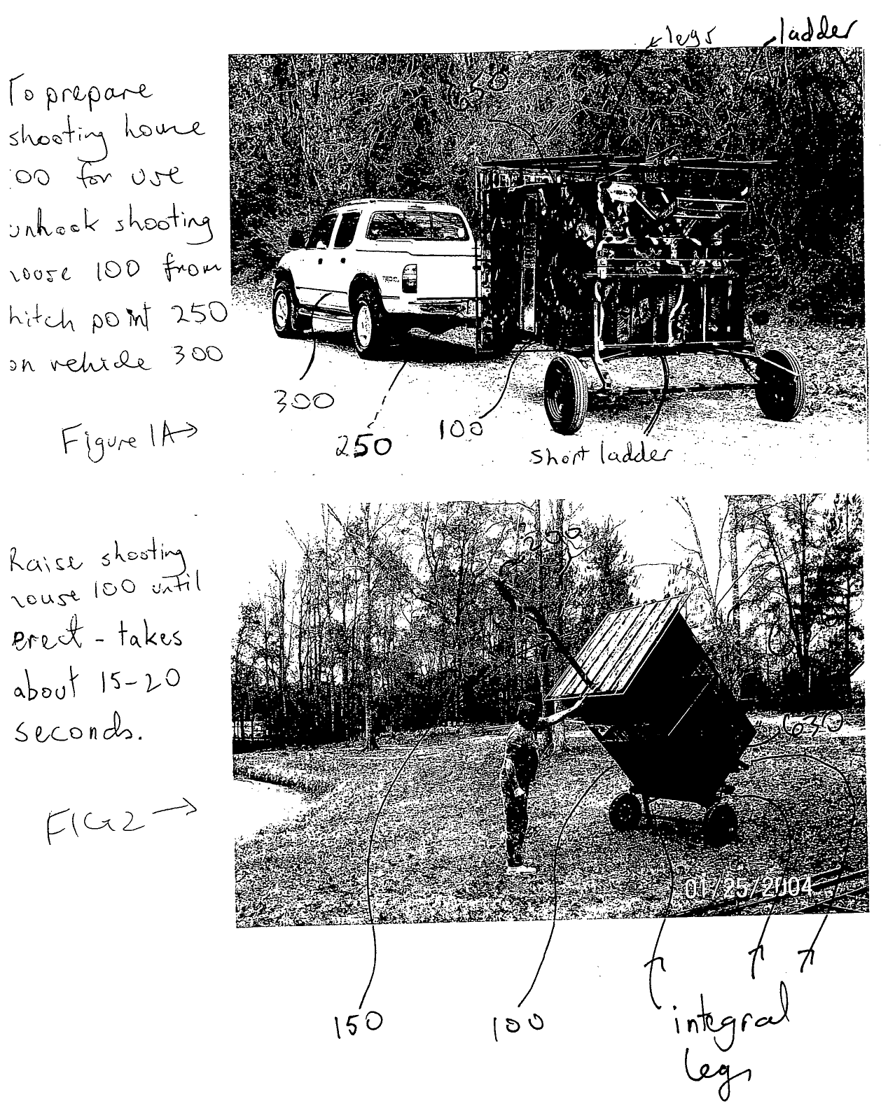

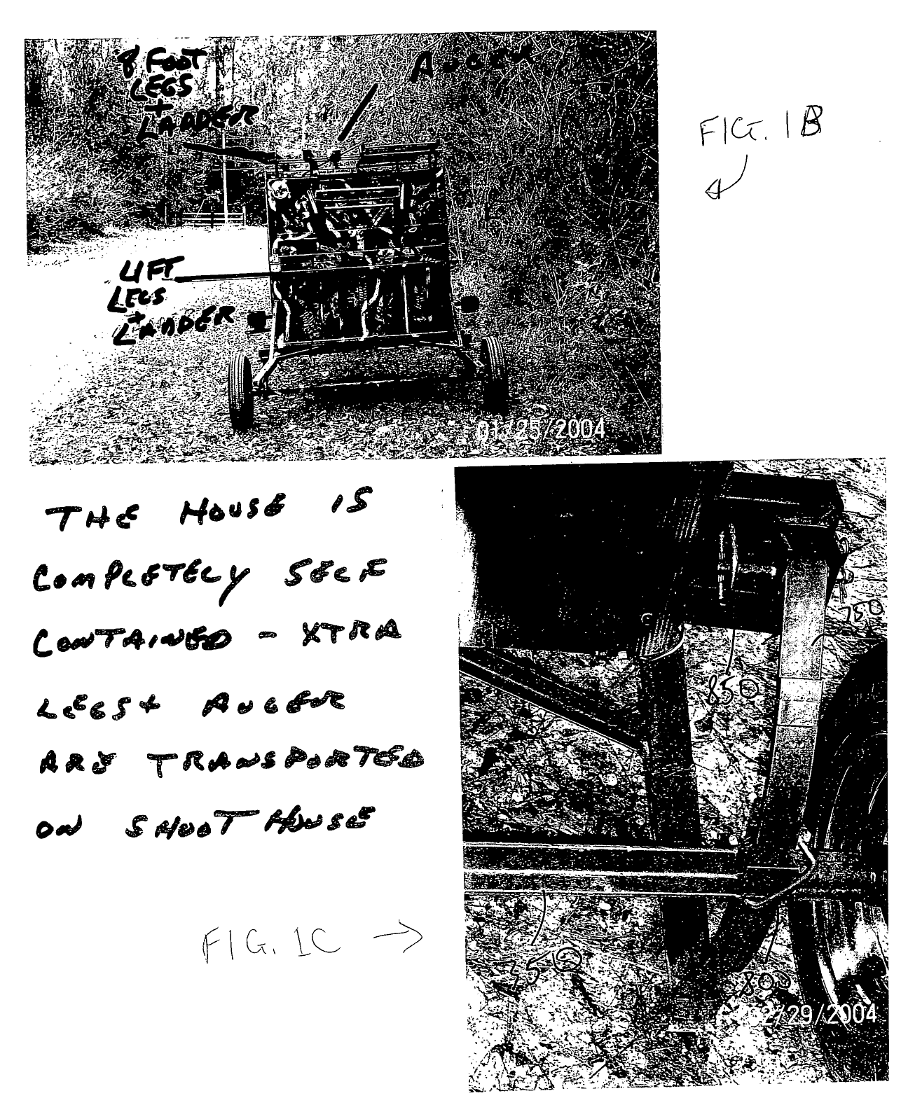

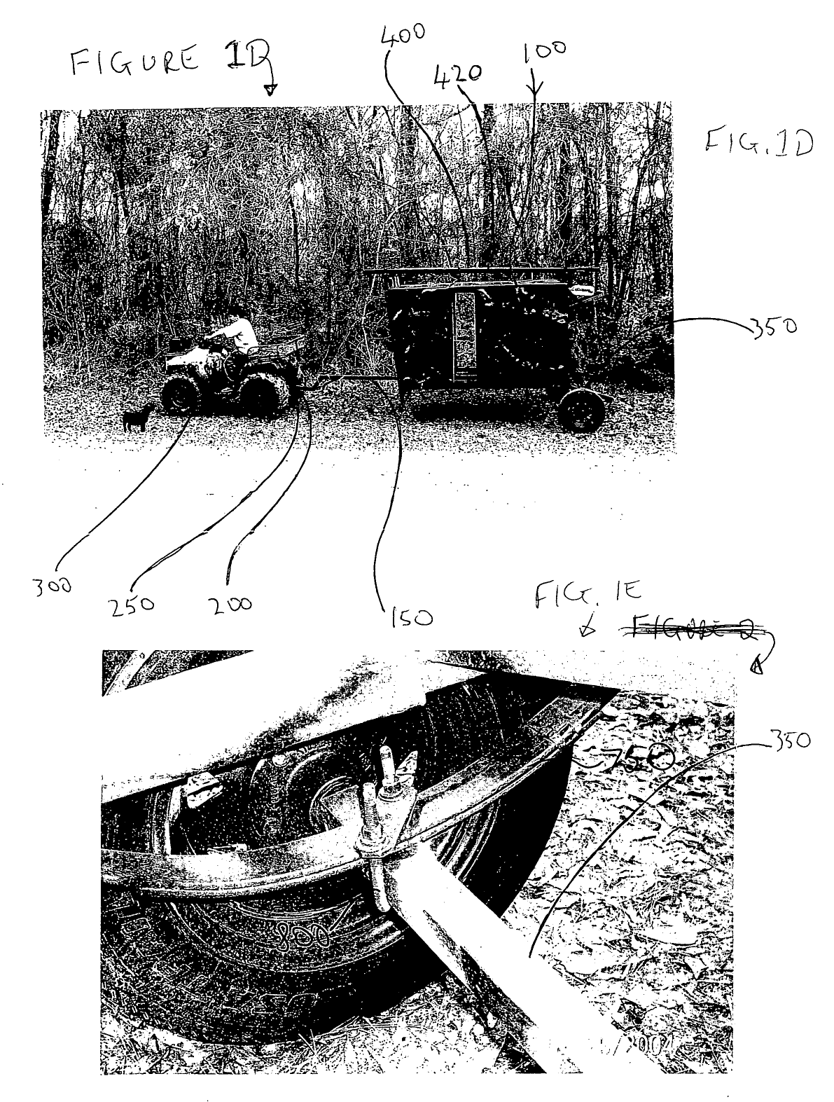

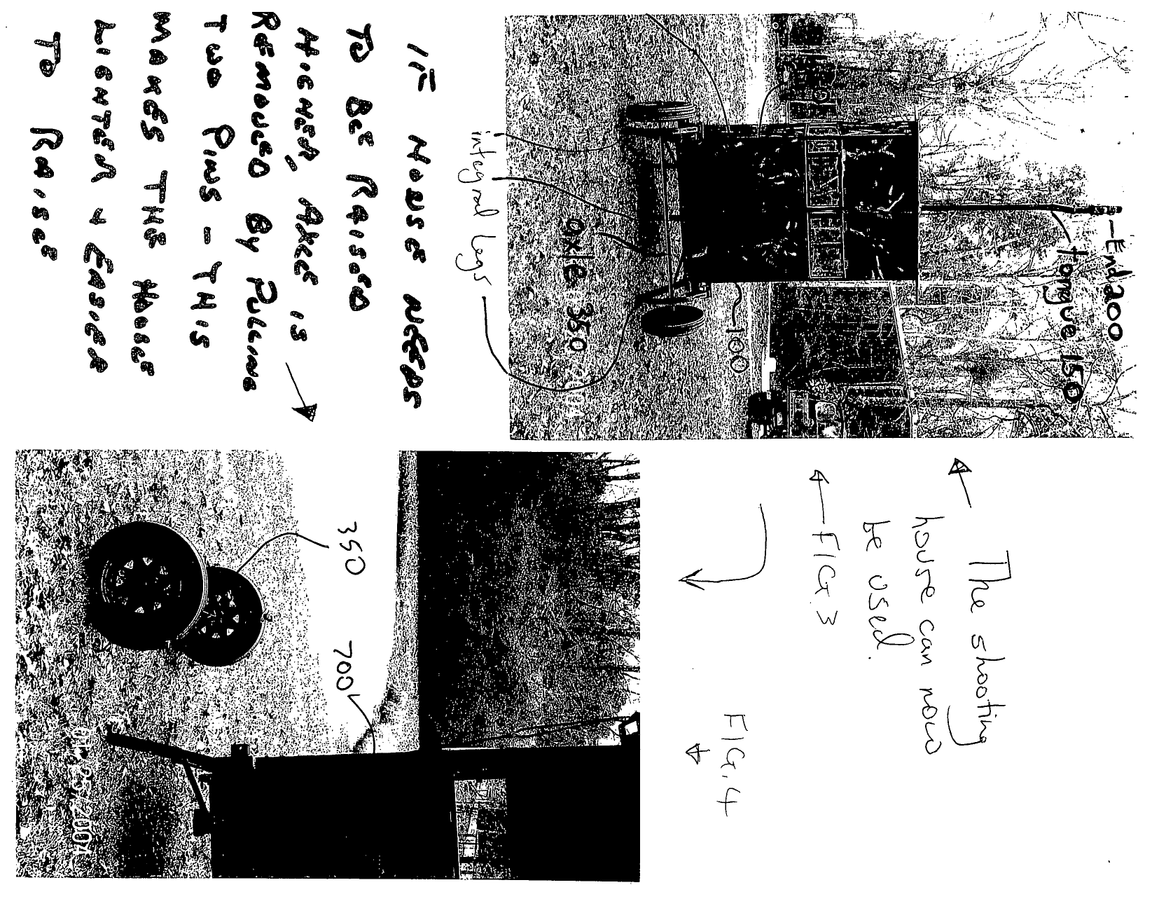

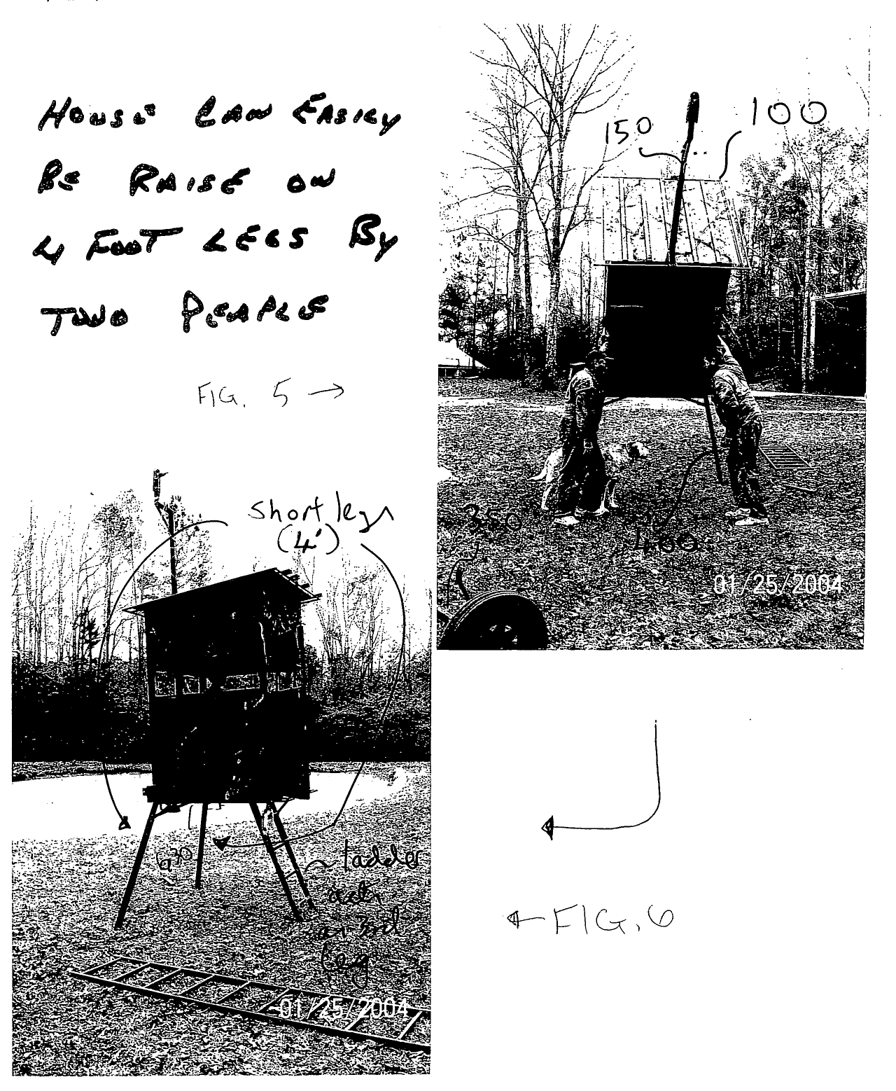

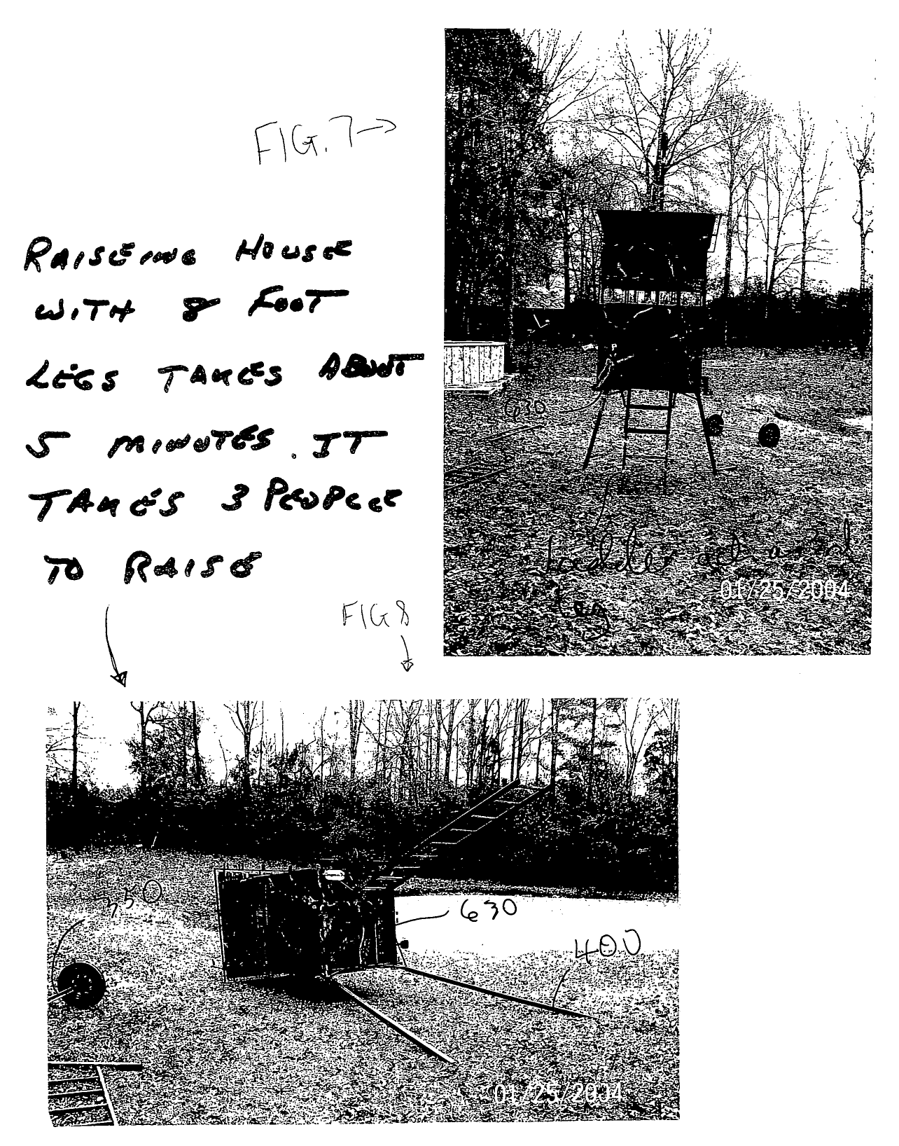

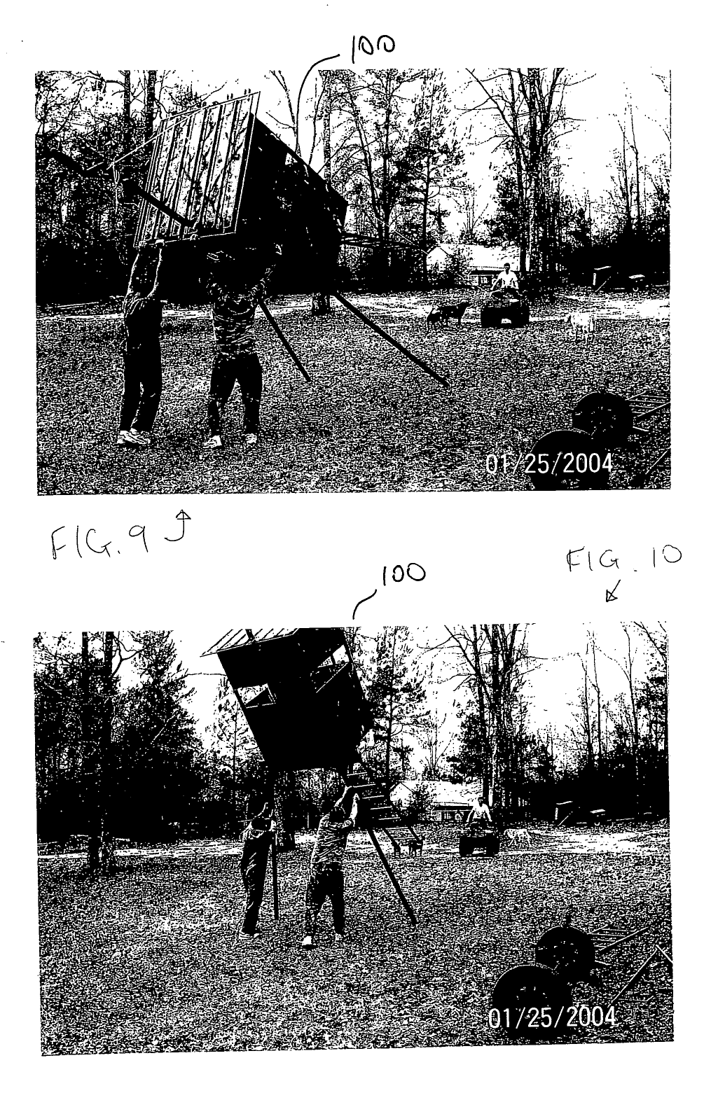

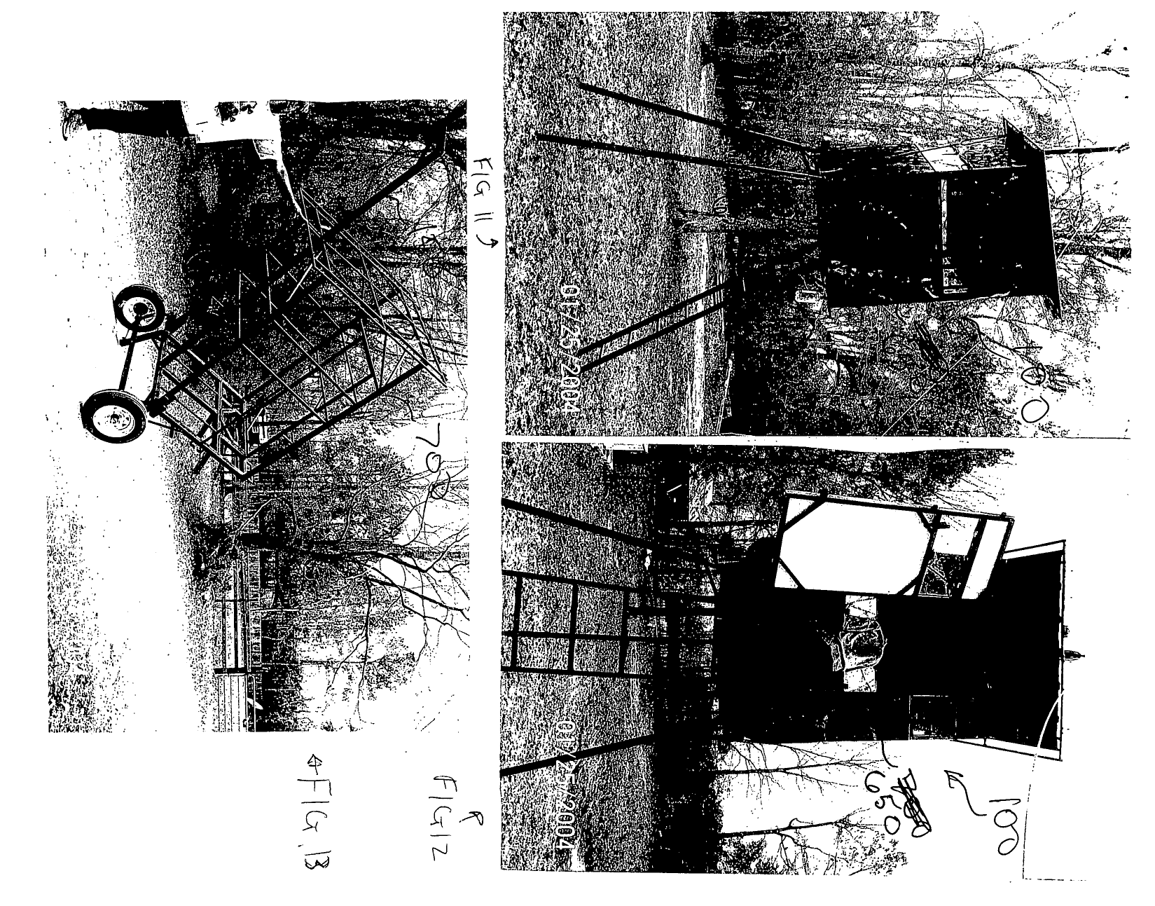

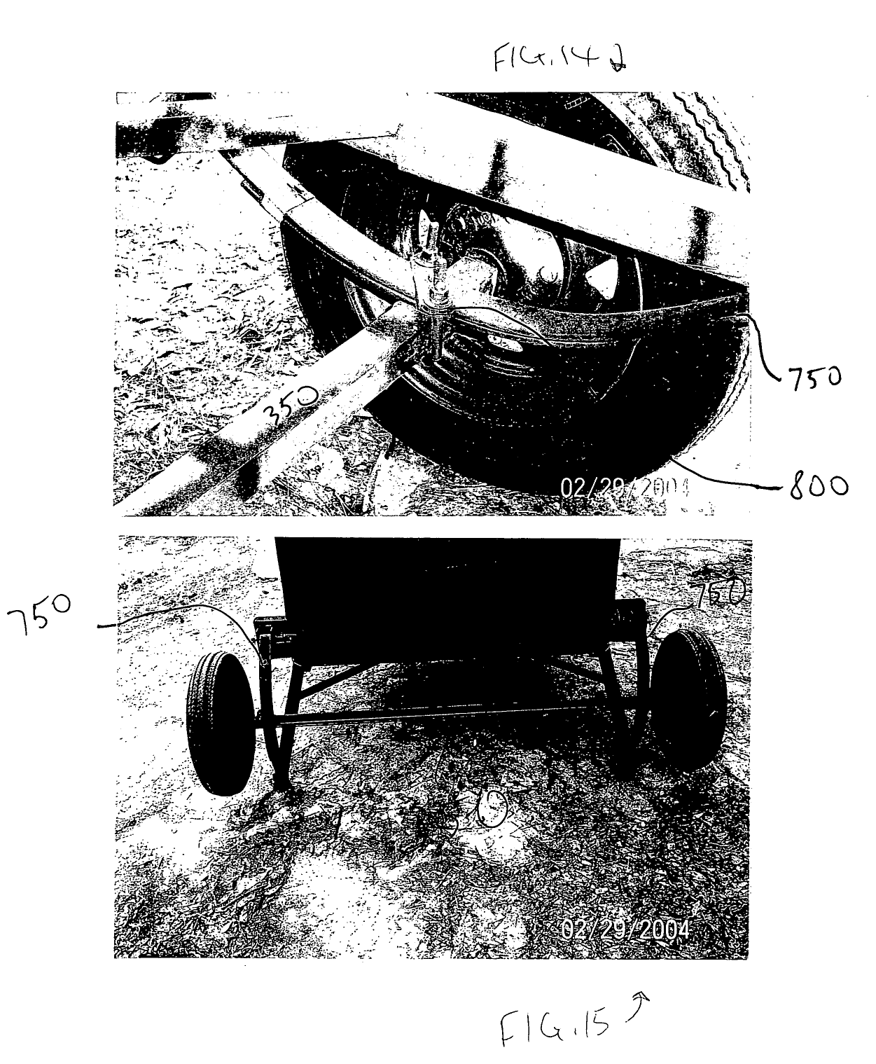

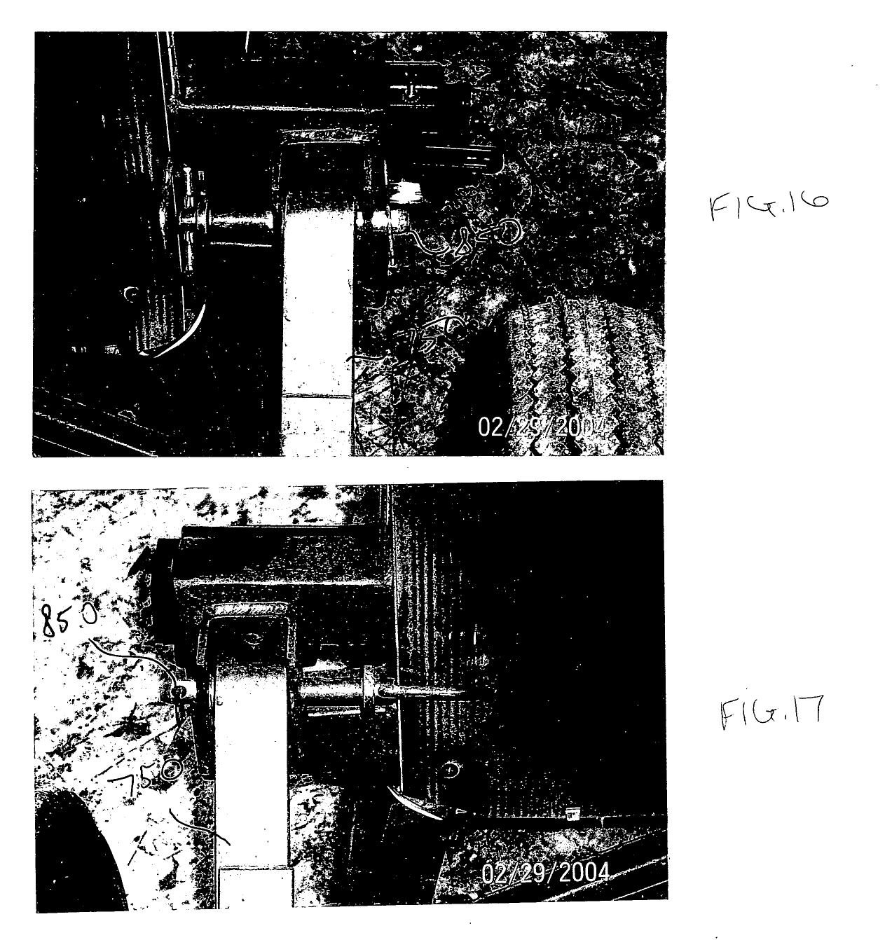

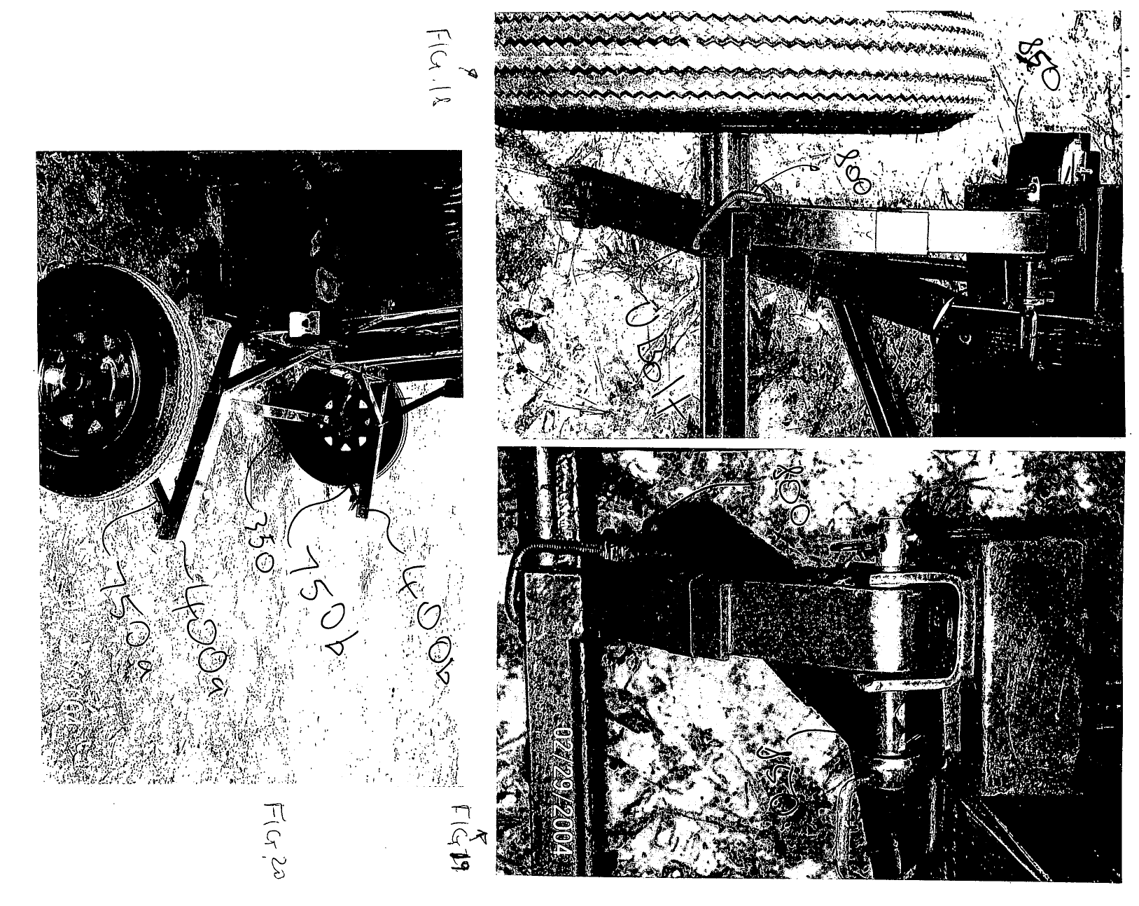

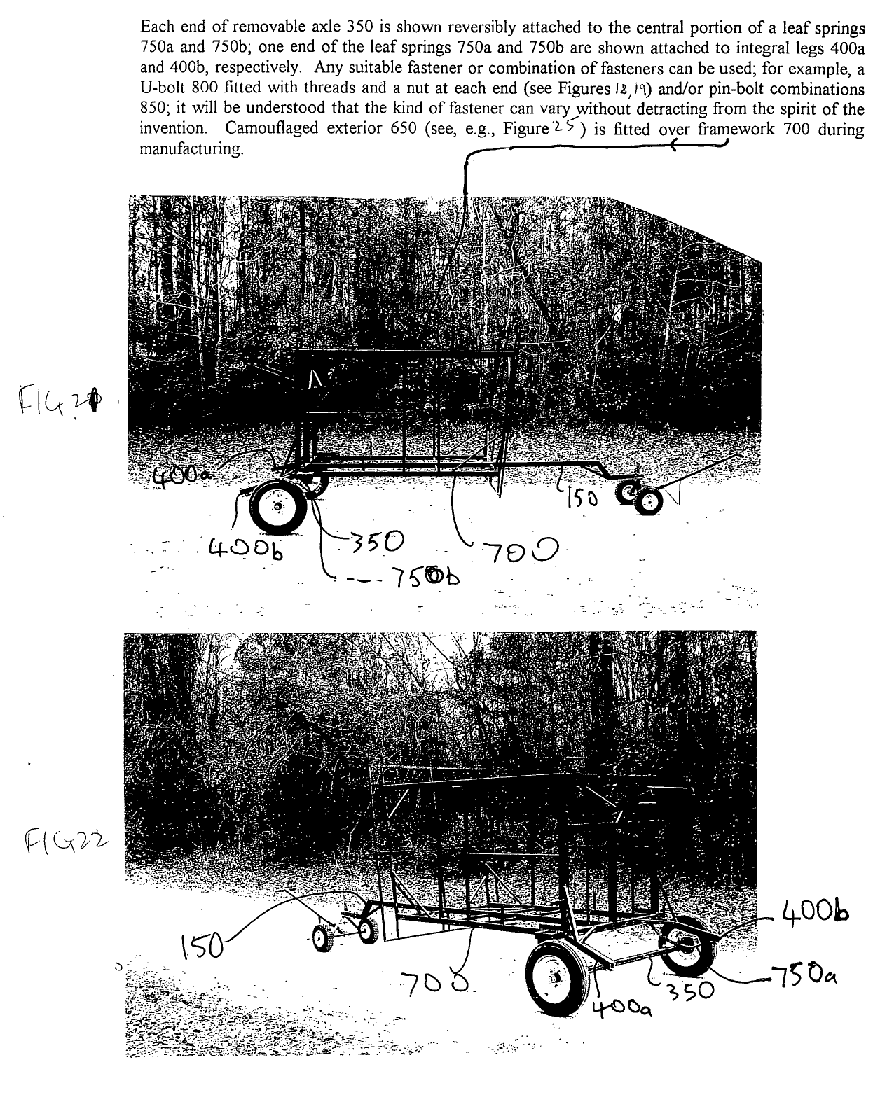

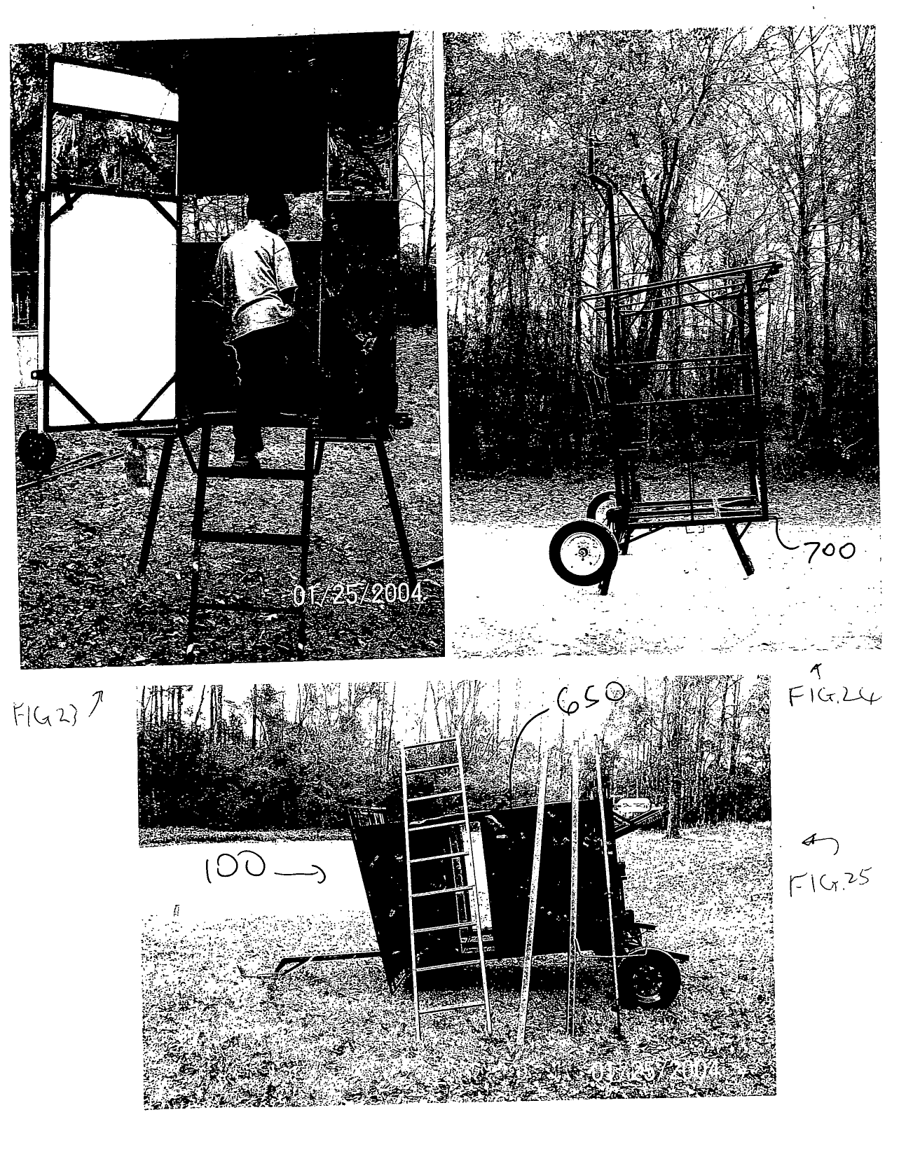

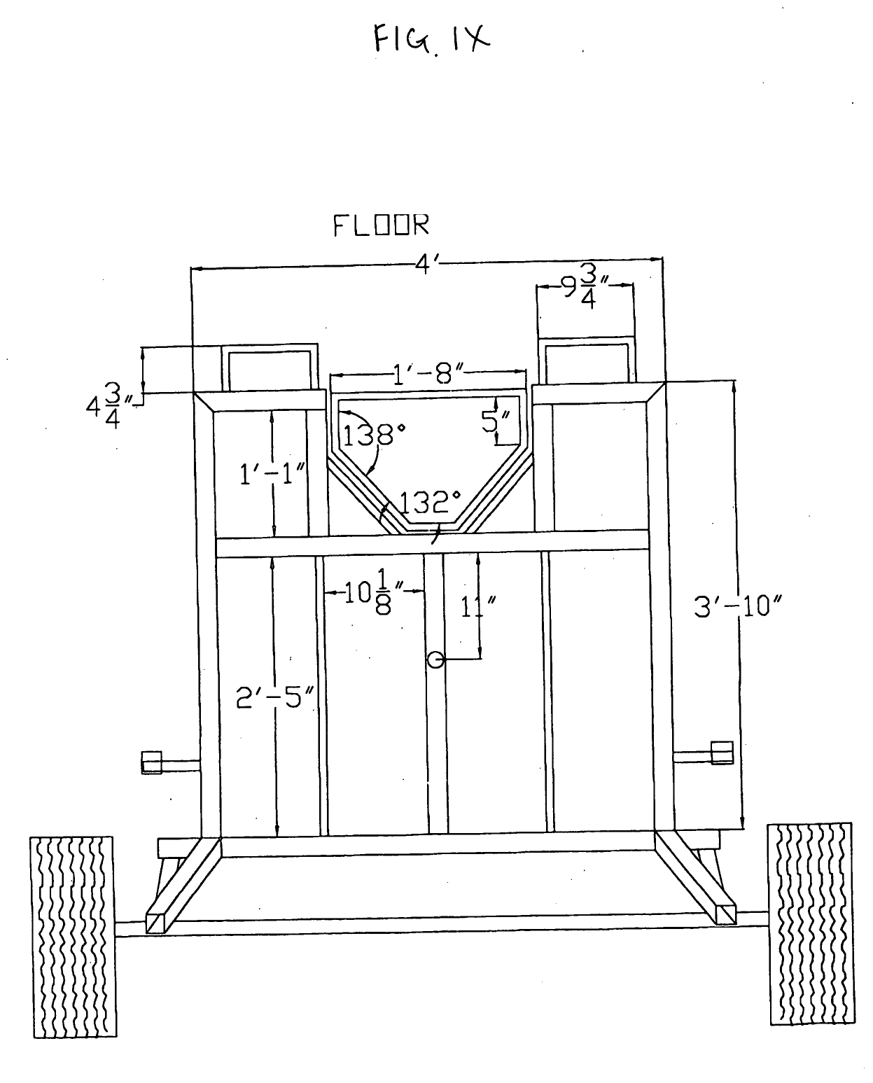

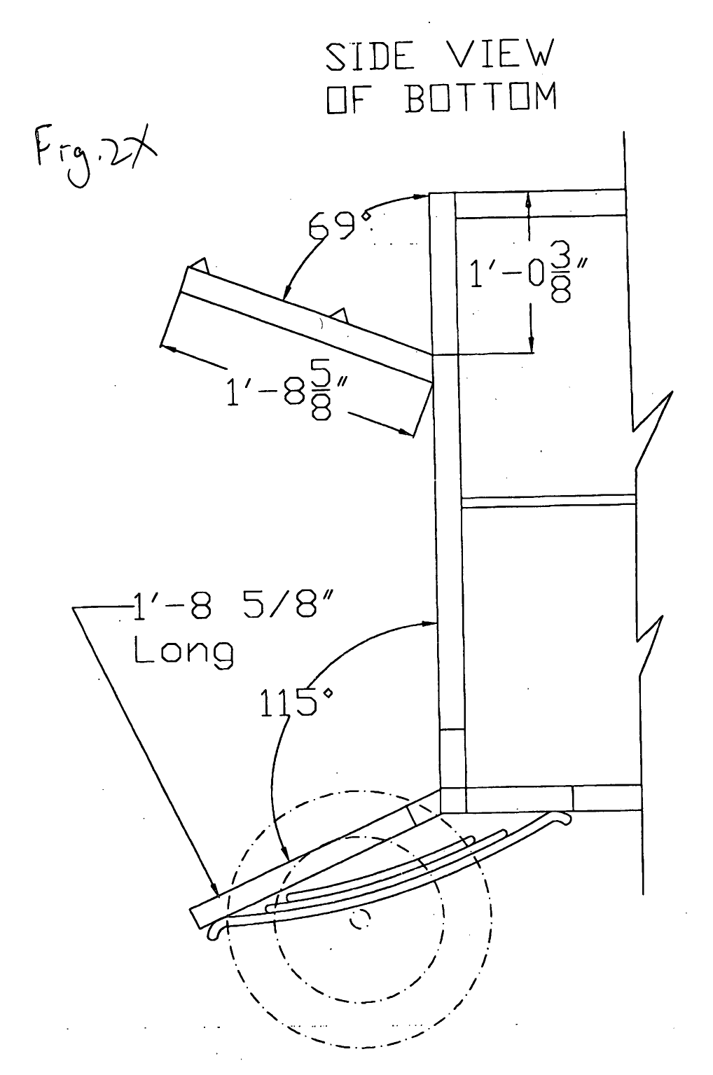

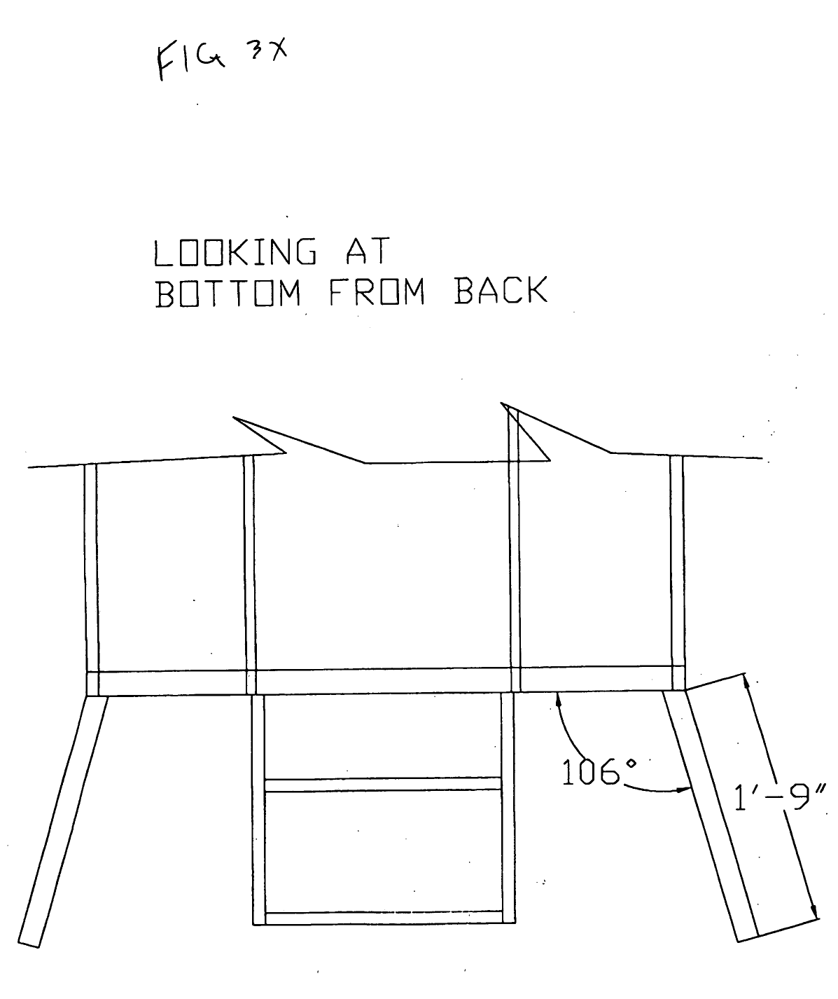

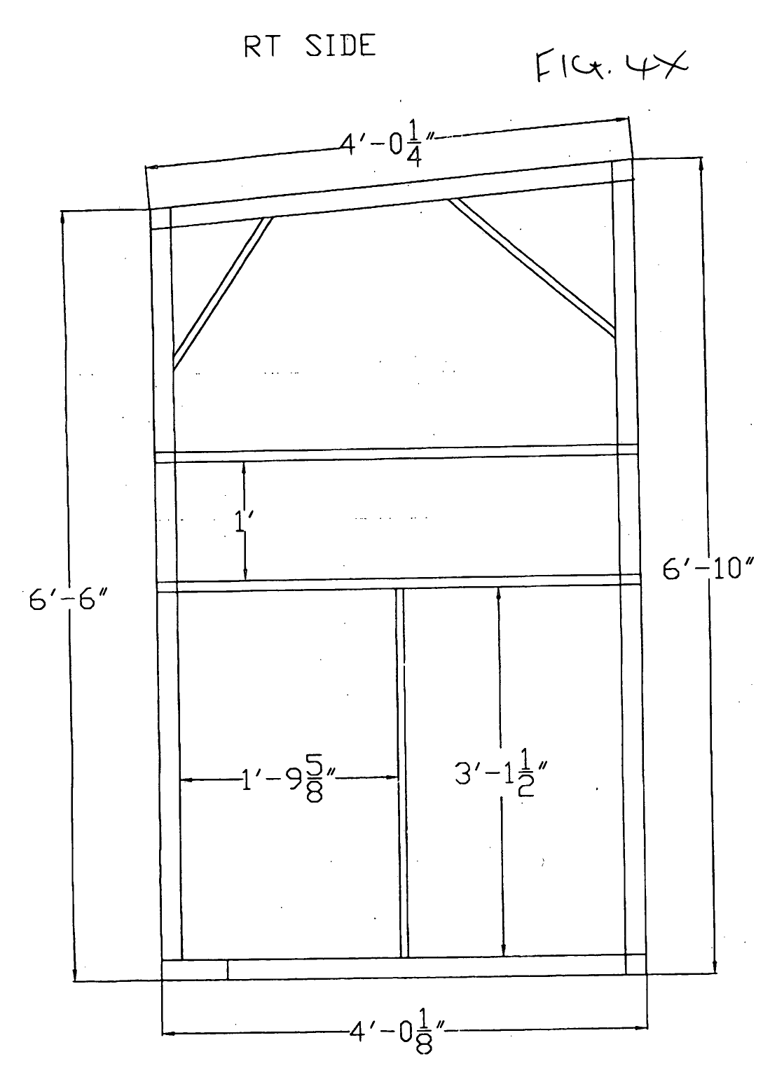

Referring to the attached FIGURES in general, and FIGS. 1A, 1B, 1C, 1D, 1E, and 2 to 13 in particular, the shooting house of the invention is denoted by the reference numeral 100 as a whole. The hunting house 100 comprises an elongated tongue 150 with one end 200 configured to attach easily to a hitch point 250 on a vehicle 300. A removable axle 350 enables the hunting house 100 to be towed; the axle 350 is typically removed and the hunting house 100 raised to an upright position ready for immediate use.

Optional sets of legs 400 and ladder 420 can be attached to the hunting house 100 to elevate the house 100 and facilitate entry thereto, respectively. The legs 400 and ladder 420 can be tied or otherwise attached to the house 100 as shown, for example, in FIG. 1. The sets of legs 400 can be of varying height such as about four and eight feet lengths; a set of integral legs (see, e.g., FIG. 2) are optionally incorporated into the design of the hunting house 100.

Referring to FIGS. A, B, C, D, E, and F, an optional anchoring system 450 can be used to anchor the hunting house 100. The preferred form of anchoring system 450 comprises at least one telescoping elongated member 500 with an auger 550 welded to one end 600 of member 500. Member 500 is preferably a two-piece telescoping pipe. The auger 550 can be replaced with any suitable drill piece so long as the drill piece can be used to anchor the device 100 to the ground.

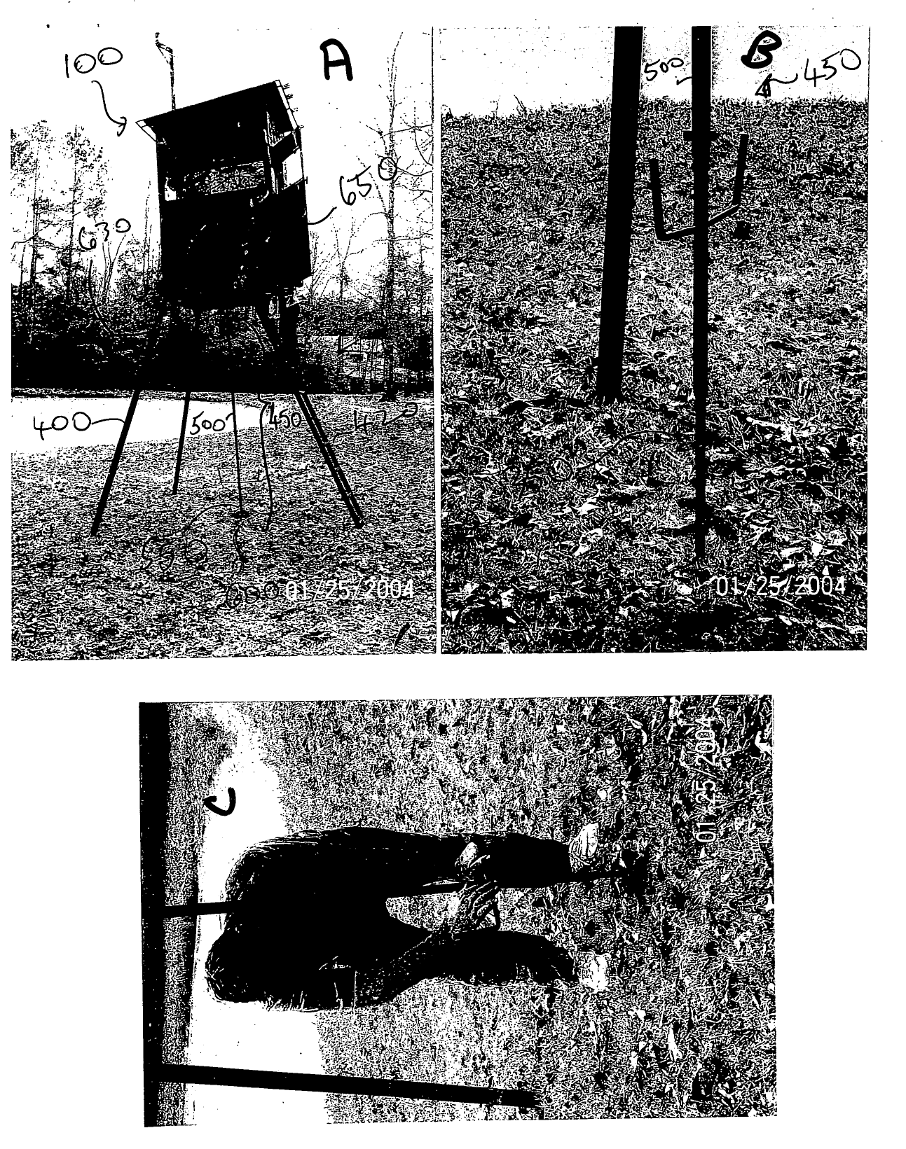

The legs 400 may be of varying length; in one embodiment a set of integral short legs are attached to the bottom 630 of the device 100 (see FIG. 2). The legs 400 may be arranged in any manner so long as the device 100 is stable; for example, the legs 400 can be arranged in the form of an incomplete tripod (see, e.g., FIG. 3) or can be attached fitted to each corner of bottom 630. Legs of varying length can be attached to the bottom 630 to raise the height of the shooting 100 above the ground (see, e.g., FIGS. 6, 8, and 11). Alternatively, the legs can be absent so that bottom 630 rests on the ground. During manufacture of the shooting house 100 a suitable exterior 650, preferably a camouflaged exterior (see, e.g., FIGS. 1 and 2), is fitted over a framework 700 (see, e.g., FIG. 21).

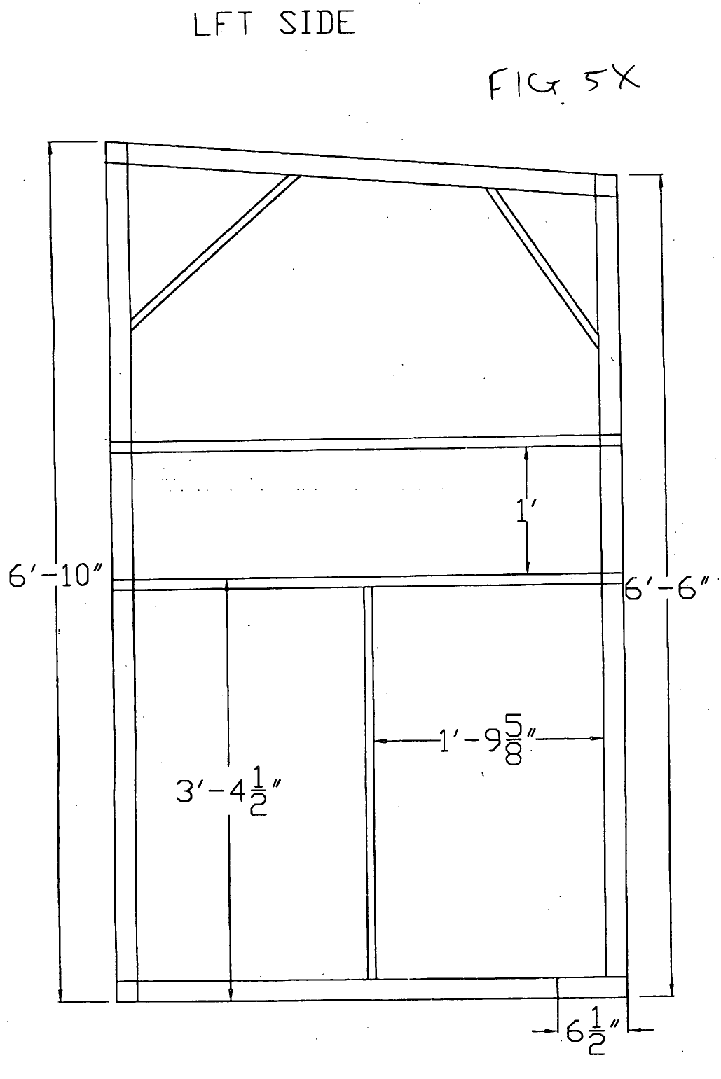

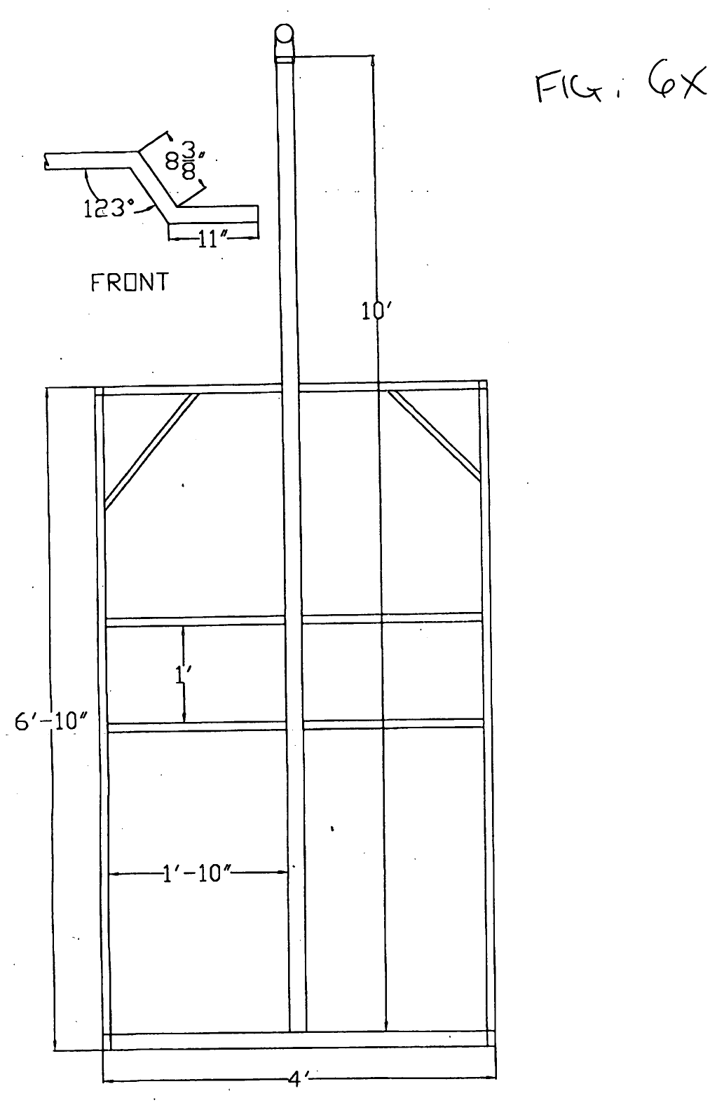

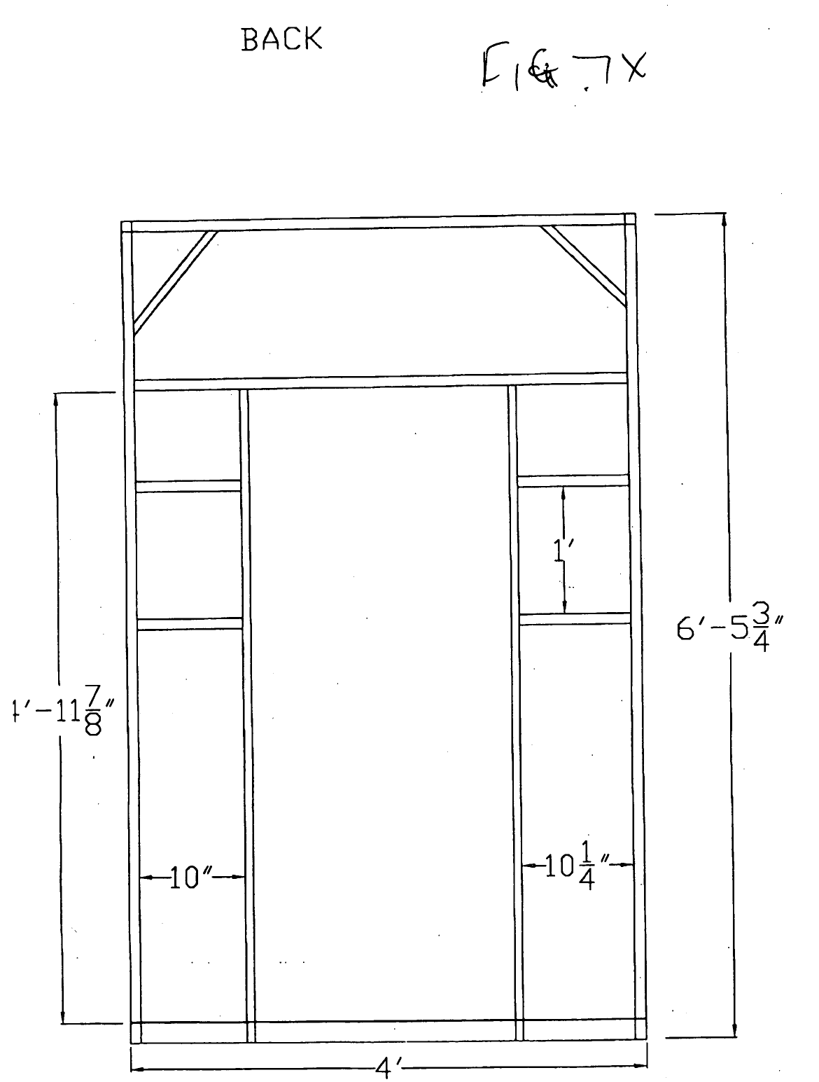

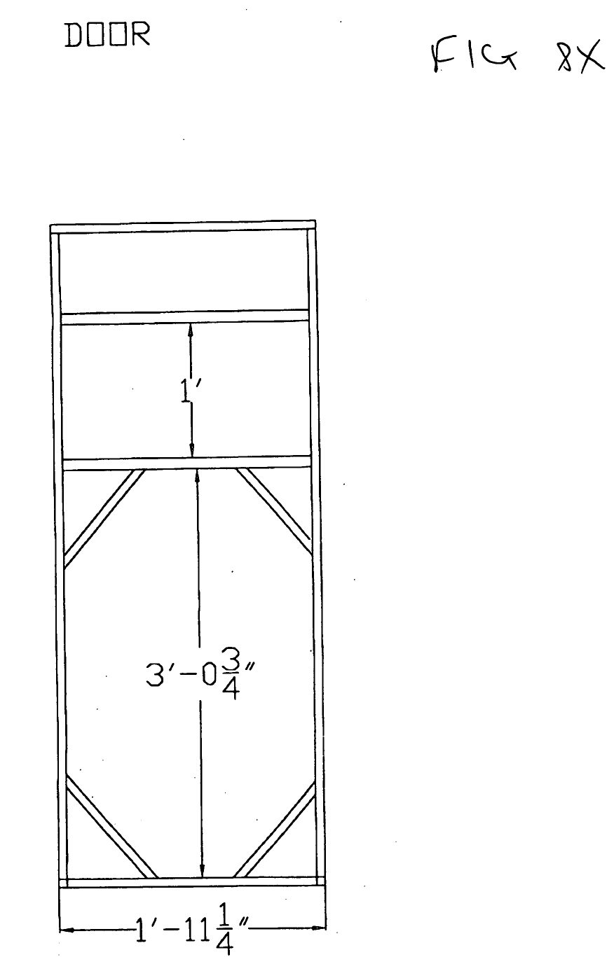

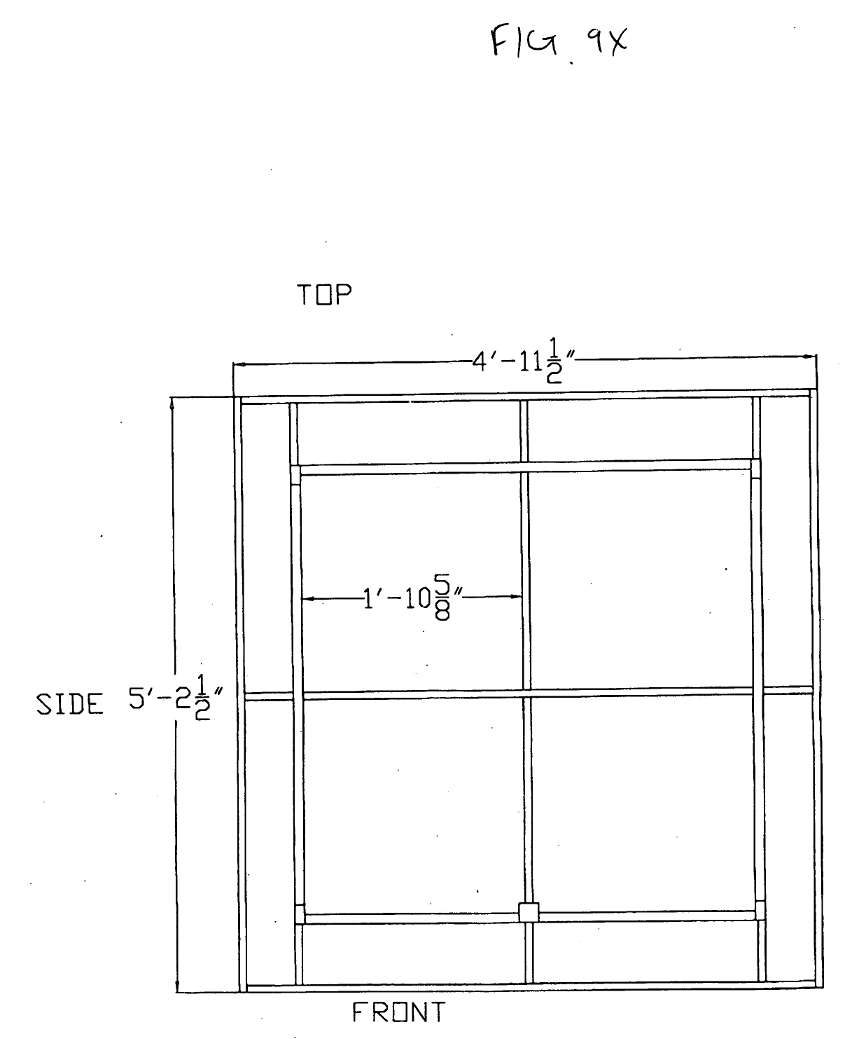

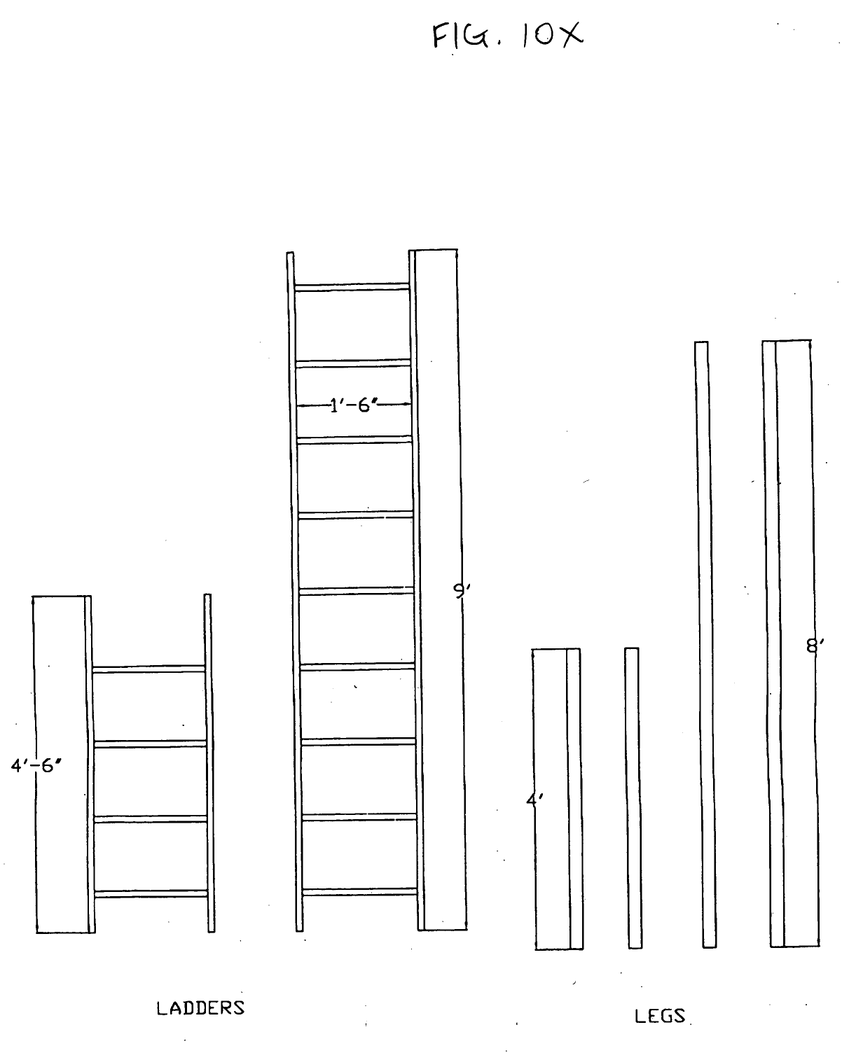

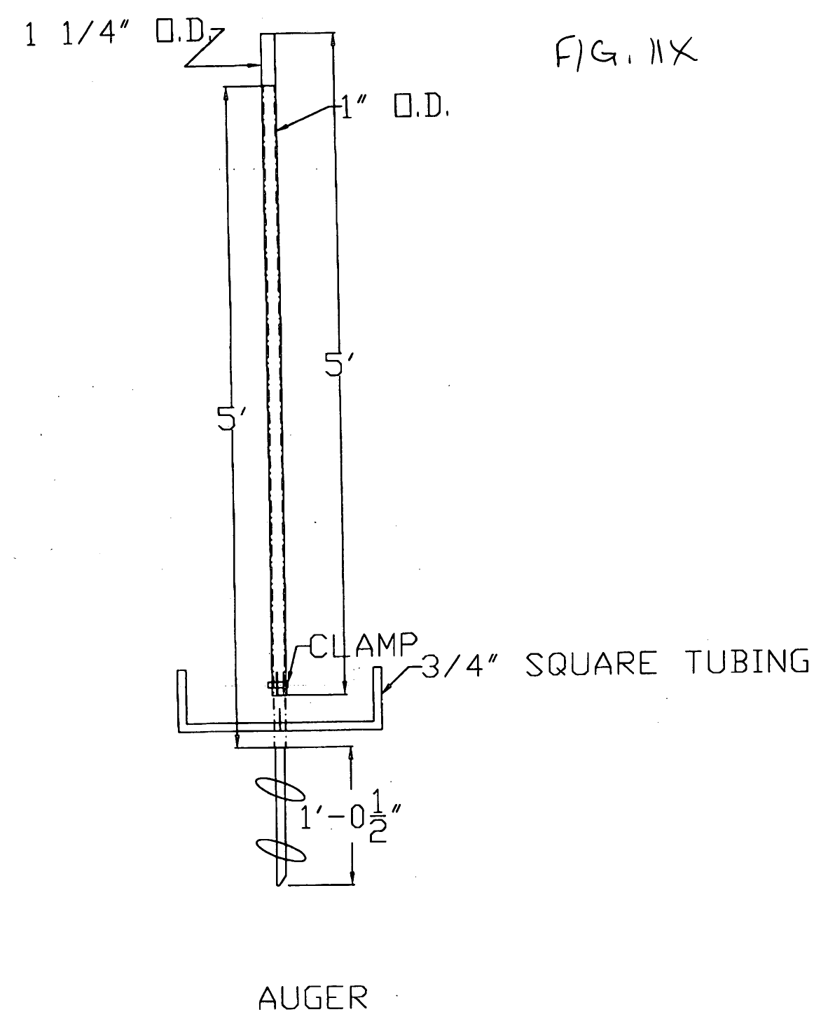

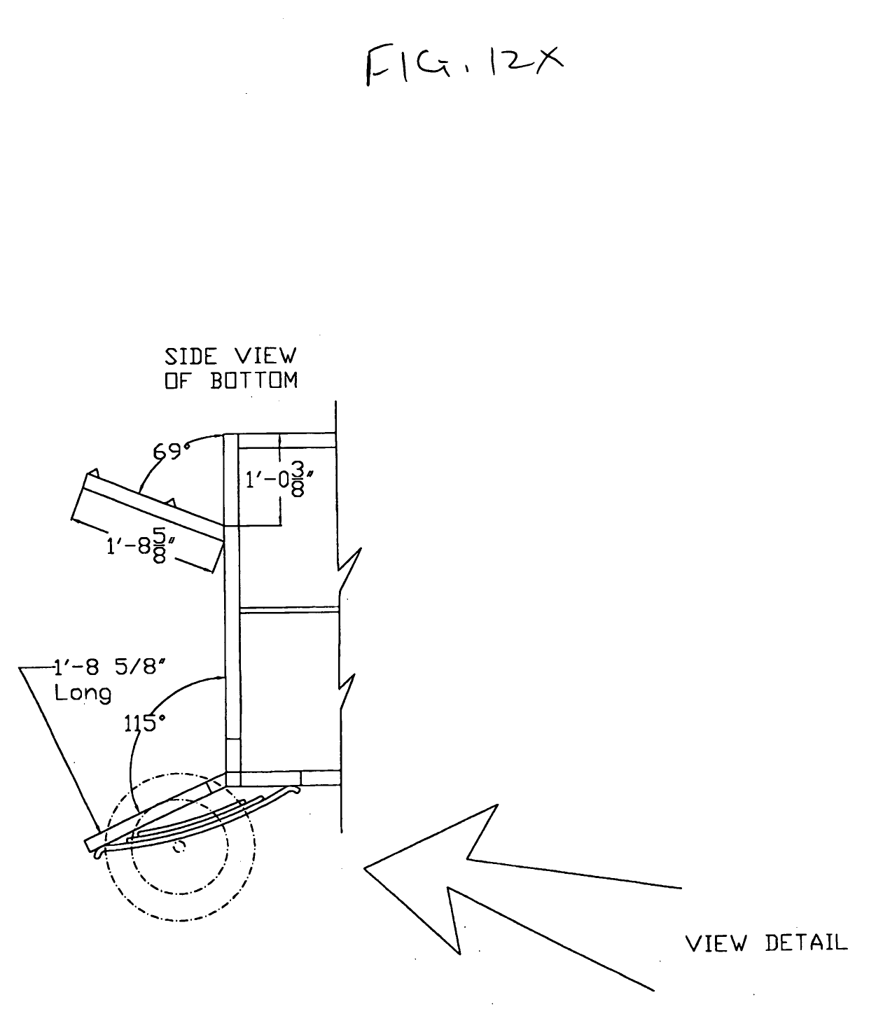

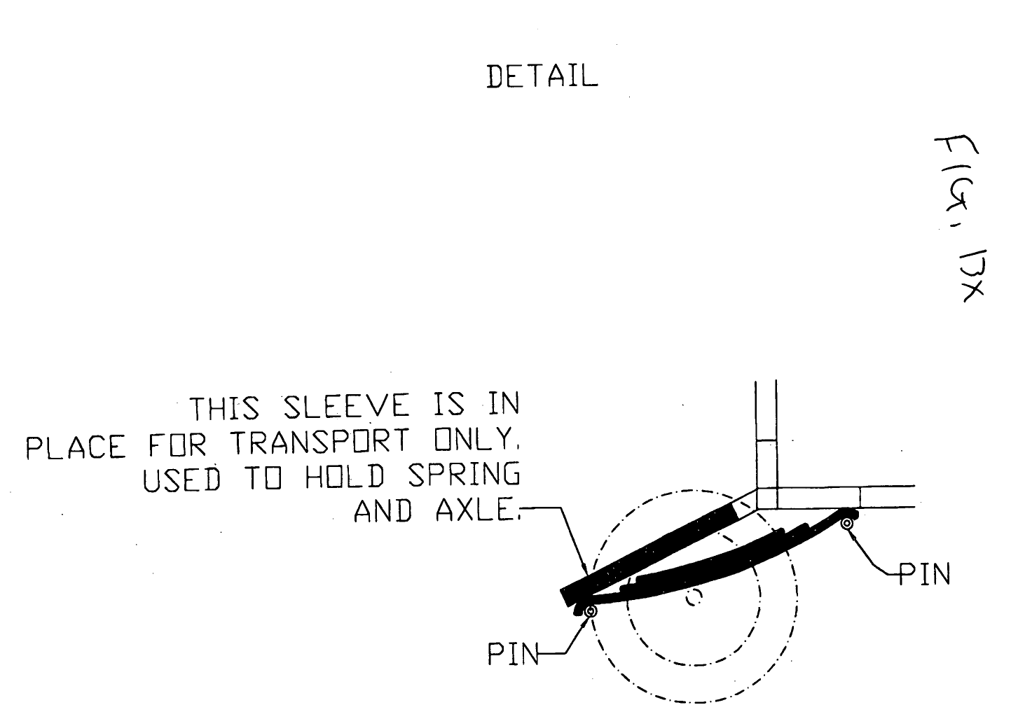

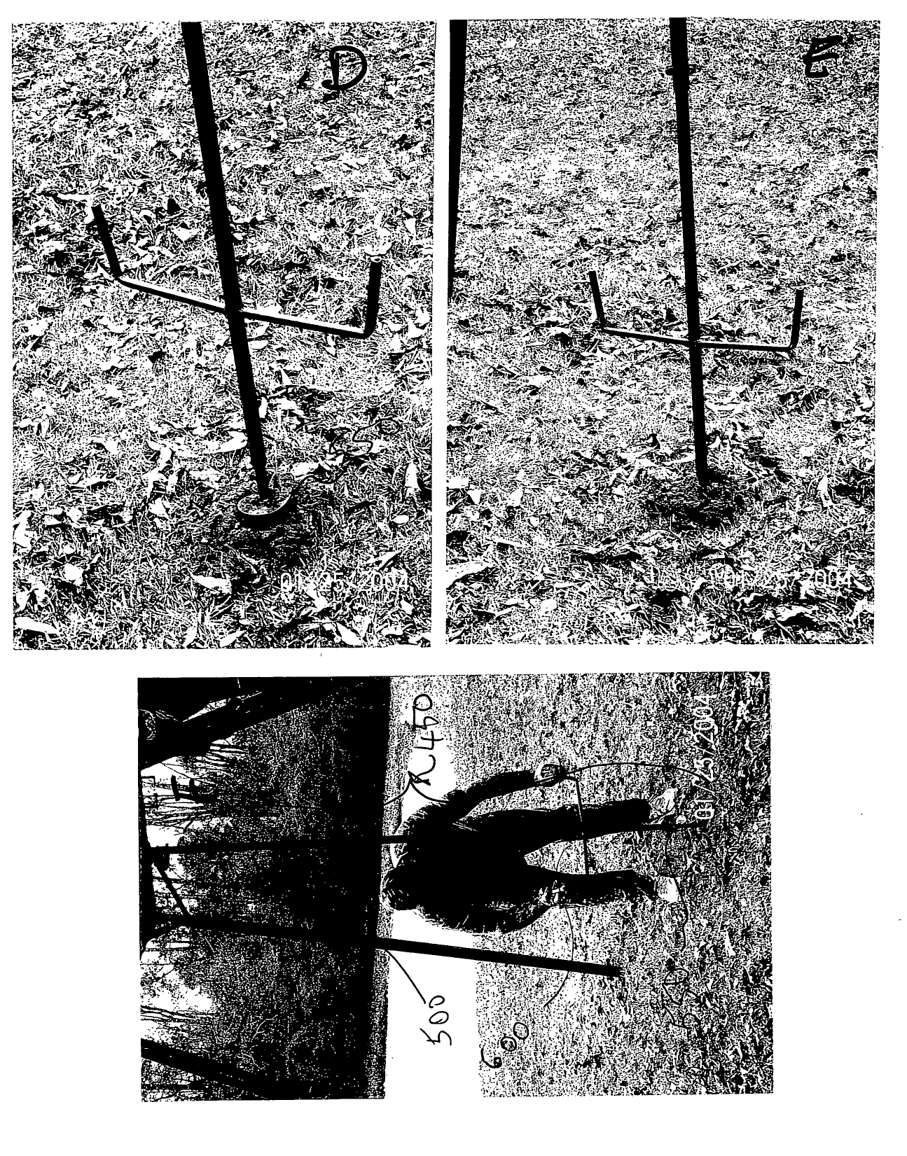

Each end of removable axle 350 is reversibly attached to the central portion of a leaf springs 750a and 750b (see, e.g., FIGS. 14 to 22); one end of the leaf springs 750a and 750b are shown attached to integral legs 400a and 400b, respectively. Any suitable fastener or combination of fasteners can be used; for example, a U-bolt 800 fitted with threads and a nut at each end (see, e.g., FIGS. 14 and 18) and/or pin-bolt combinations 850 (see, e.g., FIG. 18); it will be understood that the kind of fastener can vary without detracting from the spirit of the invention.

One embodiment of the invention is described on pages 7 to 9 and associated FIGS. 1X to 13X. With respect to this embodiment it will be understood that specific measurements and dimensions can vary; for example, while the preferred floor area of the shooting house is 4 foot by 4 foot area (i.e., about 16 square feet), the floor area is preferably in the range between about 6 square feet and about 50 square feet, and more preferably in the range between about 10 square feet and 30 square feet, and still more preferably in the range between about 10 square feet and 20 square feet.

Additional subject matter, which forms part of the Detailed Description, is found on pages 7A, 7B, and 7C. A parts list is provided on page 8. It is to be understood that the parts list is not intended to limit the invention.

It is also to be understood that the present invention is not limited to the embodiments described above, but encompasses any and all embodiments within the scope of the following claims.

My invention is intended to fill a need for a portable shoot house that could be set up in a matter of seconds. This shoot house can be towed from your home to a hunting site then set up without any tools or preparation time. It puts the hunter In a warm, dry, 4 foot by 4 foot area instantly and without the need for assembly or preparation time. Most of the time a hunter will erect his stand before daylight and will leave after dark, this Is the reason I wanted to keep this house simple and easy to erect. Some hunting management areas will not allow any stands to be left overnight, this is why I wanted to build a stand that is truly portable and can be moved in a matter of seconds.

I completed a working prototype stand in January 2004 and this is the first and only stand of it's kind that I know of.

I have built the stand with the axle as far to the rear of the stand as possible. This axle arrangement allows for the stand to be towed on the highway safely. The axle arrangement also allows the user to raise the stand by hand with a minimum amount of effort because the weight of the stand pivots on the axle. The axle comes off the stand without any tools, just pull two pins and lift off. This protects the owner from theft of the stand if he does want to leave his stand at a hunting site. Although this stand is intended to be used at ground level, I have provided a means to raise the stand on either 4 or 8 foot legs. This is another reason I wanted the axle to be taken off easily, without the weight of the axle the stand is much easier to raise.

The stand is built with square tubing, 16 gauge. It is completely welded with very few moving parts. This makes the stand very quiet when used which is important to any hunter. The roof is light gauge roofing metal screwed over foam gaskets. The frame work is covered on all four sides with PVC sign board. The signboard resembles corrugated cardboard, but is PVC plastic. This material has air pockets that both insulate the house for temperature and noise. The material I have used make the shoot house strong, lightweight, weatherproof, easily transportable and easy to set up. The strength of the steel tubing allows for minimum wall bracing, thus allowing more area for windows. This stand gives you 360 degree windows with no blind spots. Windows are ¼″ plexiglass and all open and close.

When the stand is lowered in towing position it has a low stance that makes for less wind resistance, making it tow easy and with less drag on the towing vehicle. In towing position, the stand can also be used to store and transport hunting gear, clothes and supplies.

My intentions when I started this project, were to build a lightweight, portable, dry, warm, quiet and easy to use stand. The stand construction makes is very quiet while using the stand and also while towing through the woods to hunting site.

This stand fills the need for a stand that does what I Intended it to do. It is also versatile in that it can be raised at a total of three levels, is self contained and requires no set up time at ground level and just a few minutes at other levels.

Materials

16 gauge 2×2 square tubing
16 gauge ¾×¾ square tubing
16 gauge 1×1 square tubing
16 gauge 1×2 square tubing
4 sheets of 4×8 PVC sign board
4×4 12 gauge metal roofing
½″ wafer board flooring
¼″ plexiglass windows
2-500 lb. Springs
70 inch axle
13″ wheels and tires
2″ trailer coupler
   Construction
Frame work-Welded
Roof, Floor and Siding-Screwed
Windows-Bolted w/nuts and bolts
   TOTAL WEIGHT—455 LBS.
   Parts List for

Title: Portable Shooting House

| | |
|---|---|
| 100 | shooting house 100 |
| 150 | an elongated tongue 150 with one end 200 configured to |
| | attach easily to a hitch point 250 on a vehicle 300 |
| 200 | an elongated tongue 150 with one end 200 configured to |
| | attach easily to a hitch point 250 on a vehicle 300 |
| 250 | an elongated tongue 150 with one end 200 configured to |
| | attach easily to a hitch point 250 on a vehicle 300 |
| 300 | an elongated tongue 150 with one end 200 configured to |
| | attach easily to a hitch point 250 on a vehicle 300 |
| 350 | removable axle 350 |
| 400 | Optional sets of legs 400 (includes integral legs 400a |
| | and 400b) and ladder 420 can be attached to the |
| | hunting house 100 |
| 420 | Optional sets of legs 400 (includes integral legs 400a |
| | and 400b) and ladder 420 can be attached to the |
| | hunting house 100 |
| 450 | anchoring system 450 |
| 500 | anchoring system 450 comprises at least one |
| | telescoping elongated member 500 with an Auger 550 |
| | welded to one end 600 of member 500 |
| 550 | anchoring system 450 comprises at least one |
| | telescoping elongated member 500 with an Auger 550 |
| | welded to one end 600 of member 500 |
| 600 | anchoring system 450 comprises at least one |
| | telescoping elongated member 500 with an Auger 550 |
| | welded to one end 600 of member 500 |
| 630 | Bottom 630 of hunting house 100 |
| 650 | camouflaged exterior 650 |
| 700 | Framework 700 |
| 750 | Leaf spring 750 |
| 800 | U-bolt 800, fitted with threads and a nut at each end |
| 850 | Pin-bolt combination 850 |

I claim:

1. A hunting house, comprising:
an elongated tongue 150 having first and second opposite ends, wherein one end is configured to attach to a vehicle hitch point;
a removable axle 350;
a framework 700; and
a camouflaged exterior 650 fitted over said framework 700.

2. The hunting house according to claim 1, further comprising an anchoring system 450.

3. The hunting house according to claim 1, further comprising a plurality of legs 400.
